(19) United States
(12) Patent Application Publication
(10) Pub. No.: US 2026/0013788 A1
(43) Pub. Date: Jan. 15, 2026

(54) Hand Sensation Mapping

(71) Applicant: Ultraleap Limited, Bristol (GB)

(72) Inventors: Roberto Antonio Montano Murillo, Bristol (GB); William Thierry Alain Frier, Bristol (GB); Benjamin John Oliver Long, Bristol (GB); Patricia Ivette Cornelio Martinez, Bristol (GB)

(21) Appl. No.: 19/260,546

(22) Filed: Jul. 6, 2025

Related U.S. Application Data
(60) Provisional application No. 63/669,402, filed on Jul. 10, 2024.

(51) Int. Cl. A61B 5/00

(52) U.S. Cl. CPC A61B 5/4827; A61B 5/0077

(57) ABSTRACT

An improved low-fidelity single-plane-based haptic rendering that affect the sensation designed by a sensation editor (sketch-based UI) tool is described by generating a dynamic mapping to redirect the haptic rendering over a 3D hand model in real-time. The four solutions comprise (1) the generation of a bijective mapping between the template (2D) and hand spaces (3D), (2) a direct skinning approach for sensation relocation in 3D, (3) a direct 3D to 3D mesh mapping, and (4) a smooth-blend skinning directly to the sensation points. This allows a playful action which continuously provides feedback to the user as they progress along their actions-in this case the object becoming “squished” as they select it, and “unsquished” as they summon it.

### PRIOR APPLICATIONS

This application claims the benefit of the following application, which is incorporated by references in its entirety: U.S. Provisional Patent Application No. 63/669,402, filed on Jul. 10, 2024,

### FIELD OF THE DISCLOSURE

The present disclosure relates for improving low-fidelity single-plane-based haptic rendering issues that affect the sensation designed by a sensation editor (sketch-based UI) tool

### BACKGROUND

Currently, designing mid-air haptic sensations can be done in two main ways:
1. By manually adjusting the settings to generate a haptic pattern.
2. By drawing a 2D sketch inside a UI in which the user would define not only the set of positions but intensity variations along the pattern in a simple visual manner.

The haptic sensation design using manually generated settings is highly complex as it requires the user to master different skills from programming to physics. The UI sketch-based approach is an easier way for naïve users to design haptic patterns [1], and therefore, the best option among these two for the customers of this technology.

However, this type of tool commonly uses 2D templates limiting the rendering to custom haptic sensations in 2D space, and therefore, the design of 3D sensations is not possible. This means that a sensation that is rendered on the user's hand, assumes a 2D plane (which contains the designed sensation in 2D), and anchors the target location on the hand through a fixation point (e.g., palm's center).

For example, assuming a circular haptic sensation that targets the fingers with the center on the palm, the sensation will be perfectly displayed when the user has an open hand pose. However, performing a fist hand pose during the sensation rendering will move all the fingers out of the sensation plane, causing the haptic rendering to miss the target fingers. Similarly, assuming a sensation anchored on the palm center that moves up towards the fingers, the abduction/adduction deviation of the fingers is not supported. This means that a simple haptic line linking the palm center to the index fingertip will often miss the finger.

Current method issues include:
1. The current 2D rendering method is constraining the sensation size to local areas (finger's phalanges or palm area). That is, it does not allow the correct display of sensations moving across fingers or across fingers and palm when the hand is not fully open or when the fingers are moving.
2. There is a rendering mismatch between the designed and perceived sensation due to the unrealistic representation of the rendering plane using the 2D template. The current 2D perspective of the hand does not accurately represent how the sensation will be displayed on the real hand increasing pattern re-design and increasing overall design time (probably impacting the user's experience).
3. There is a lack of support for designing haptics feedback on body parts beyond the palm (e.g. back of the hand, knuckle, forearm or face).

The current method to overcome the low-fidelity plane-based haptic rendering issues includes:
1. Anchoring the sensation design to a specific location on the hand using fixation points (e.g. fingertip, palm etc.).
2. Limiting the sensation design complexity and size. This is to keep the sensation as close as possible to the anchor point and inside the anchored area.

This solution can work for sensation designs whose sizes fit into the anchored area, for instance:
1. Display a sequence of pulses on the index fingertip.
2. Display haptic patterns on the palm. This is the area most commonly used as it is a relatively big flat area that can handle relatively large patterns.

However, this solution does not cover interdigital sensation designs, nor support designs on knuckles or the back of the hand. This solution also constrains the exploration of more complex haptic patterns.

This issue limits designers' ability to design what they want to transmit through the haptic sensation and limits it to what the UI allows them to do.

### SUMMARY

This application proposes methods to improve the low-fidelity single-plane-based haptic rendering issues that affect the sensation designed by a sensation editor (sketch-based UI) tool. By generating a dynamic mapping to redirect the haptic rendering over a 3D hand model in real-time. The four solutions comprise (1) the generation of a bijective mapping between the template (2D) and hand spaces (3D), (2) a direct skinning approach for sensation relocation in 3D, (3) a direct 3D to 3D mesh mapping, and (4) a smooth-blend skinning directly to the sensation points.

This invention differs from previous attempted solutions in that it provides a progressive way to both select, and summon an object, through the metaphor of Squishing, rather than a discrete action.

This allows a playful action which continuously provides feedback to the user as they progress along their actions-in this case the object becoming “squished” as they select it, and “unsquished” as they summon it, although other visualizations are possible as discussed above.

This progressive feedback is especially powerful as it can be paired with pose detection, which, if the user understands the pose, they need to make in order to interact with the object, intuitively suggests to the user when the object they are interacting with will be selected or summoned. This also allows the entire action to be performed in one fluid motion-moving into, and out of, a pose.

### BRIEF DESCRIPTION OF THE DRAWINGS

The accompanying figures, where like reference numerals refer to identical or functionally similar elements throughout the separate views, together with the detailed description below, are incorporated in and form part of the specification, serve to further illustrate embodiments of concepts that include the claimed invention and explain various principles and advantages of those embodiments.

FIG. 1 shows prior examples of haptic rendering method based on a single rendering plane anchored to the palm center.

FIG. 2 shows sequences of positions.

FIG. 3 shows the areas/planes defined across spaces.

FIG. 4 shows a cylindrical coordinate system and projection.

FIG. 5 shows a 2D texture of a hand wrapped on the 3D hand model

FIG. 6 shows a first 3D model as a haptic template.

FIG. 7 shows a second 3D model as a haptic template.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

The methods proposed in this document target to improve the transition/mapping from the sensation designed by the sensation editor (sketch-based UI) tool to generate a dynamic haptic rendering over a 3D hand model in real time. Improving the rendering fidelity and extending the available areas used to display haptic sensation on the hand.

This document considers the following solutions:
1. The generation of a bijective mapping between the template (2D) and hand spaces (3D).
1.a The generation of the 2D to 3D mapping based on a multi-space approach (Cuboid approximation).
1.b Cylinder approximation for sensation relocation in 3D based on multi-space mapping.
2. A direct skinning approach for sensation relocation in 3D.
3. A direct 3D to 3D mesh mapping.
4. Apply a smooth-blend skinning directly to the sensation points.

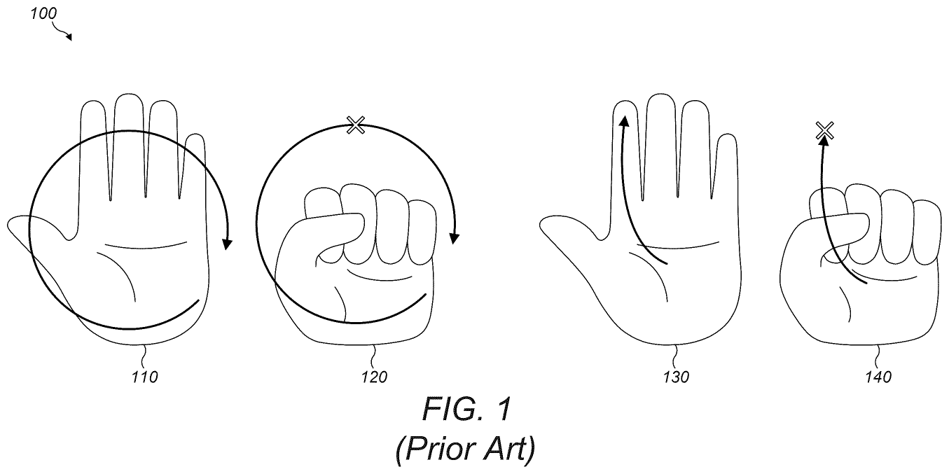

Turning to FIG. 1, shown is a prior art schematic 100 of examples of the current haptic rendering method based on a single rendering plane anchored to the palm center. The figures on the left show a haptic circle rotating across the fingers in an open hand pose 110 and missing the fingers when in a fist hand pose is done 120. The figures on the right shows a haptic point moving from the palm center to the index fingertip in open 130 and fist-hand 140 poses.

### 1. Solution 1: Generation of a Bijective Mapping Between the Template (2D) and Hand Spaces (3D)

This mapping is based on a tree of transformation matrices of both spaces. This already allows the transition from a single 2D plane (current default in the sensation editor tool) to a dynamically located multi-space haptic rendering.

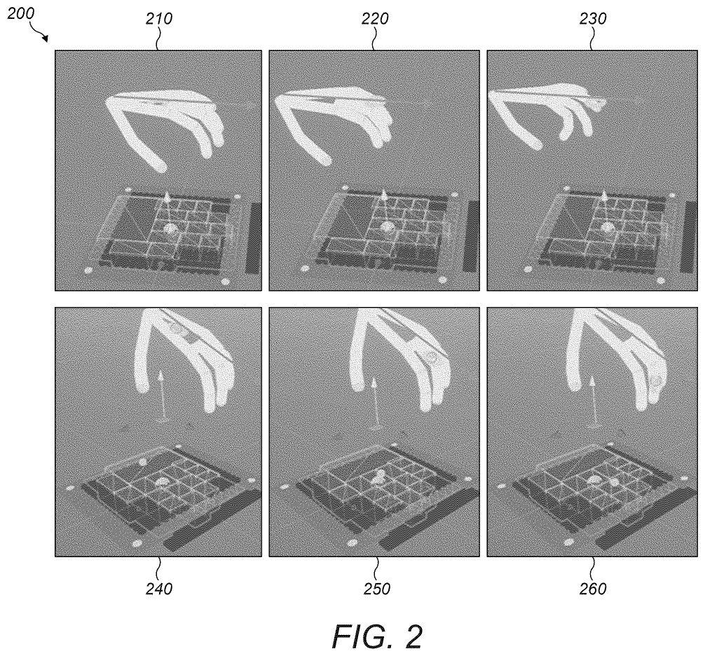

Turning to FIG. 2, shown is a schematic 200 of a top sequence of positions of a circle moving from the palm 210, through the proximal 220 and intermediate 230 phalanges of the middle finger using the current sketch-based sensation editor approach (single plane). On the bottom, the same sequence of position using the approach proposed in this document (2D to 3D sensation mapping) to relocate the position of the sensation on the correct target phalange 240 250 260.

### 1.a The Generation of the 2D to 3D Mapping Based on a Multi-Space Approach (Cuboid Approximation)

This section describes the method to generate dynamic motion retargeting using the multi-space concept.

This mathematical description uses the right-hand systems of reference, homogeneous coordinates (i.e., 3D points in A′s coordinates as p_{A}(x, y, z, 1)∈ [symbol] 4) and homogeneous transformation matrices

$$( {M}_{B}^{A}$$

∈ [symbol] 4×4, to convert coordinates from A to B).

In the first solution, the haptic sensation rendering on the hand assumes a direct correspondence between the template space (Template), where the sensation player replicates a sensation pattern from a sensation file in JSON format (from the sensation editor tool); and the Leap Motion space (LeapM), local to the haptic device. All points are mapped from one space to another directly through a transformation matrix like:

$${p}_{LeapM} = {M}_{LeapM}^{Template} * {p}_{Template} \qquad ( 1 )$$

This approach uses a set of volume pairs, one defined in each space, Template and LeapM. Let V_{Template}={a_{Template}, b_{Template}, c_{Template}, d_{Template}}⊆Template and V_{LeapM}={a_{LeapM}, b_{LeapM}, c_{LeapM}, d_{LeapM}}⊆LeapM be a volume pair described by the template coordinates and retargeted leap motion coordinates, relative to the Haptic device's center.

The transformation matrices allow us to directly map any template point p_{Template} inside V_{Template} to its analogous Volume V_{LeapM}, by computing its local coordinates in V_{Template} and mapping the point to the same coordinates in the equivalent volume VLeapM:

$${p}_{LeapM} = {( {M}_{World}^{LeapM} )}^{- 1} * {M}_{World}^{Template} * {p}_{Template} \qquad ( 2 )$$

By using this mapping strategy, the pair {V_{Template}, V_{LeapM}} now identifies two equivalent volumes in Template and LeapM spaces, even if their shape is different. Thus, not only physical vertices {a_{Template}, b_{Template}, c_{Template}, d_{Template}} are mapped to their equivalent retargeted vertices {a_{LeapM}, b_{LeapM}, c_{LeapM}, d_{LeapM}}. Any other point inside V_{Template} can also be mapped to its equivalent in V_{LeapM}.

### 1.1 Bounding the Space: Template and Leap Motion Trees

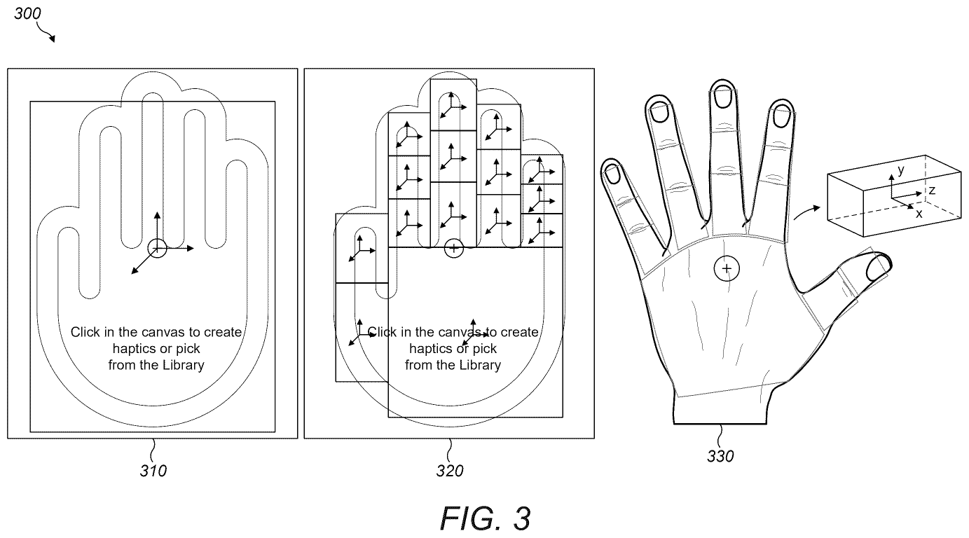

Turning to FIG. 3 shown is a schematic 300 of how the areas/planes are defined across spaces, and how muti-planes and 3D hand areas match. On the left 310 shown is a single plane template from sketch-based sensation editor approach. In the center 320 shown is a multi-plane approach as proposed in our first solution. On the right 330 shown is a 3D hand in Leap Motion coordinates.

Having a proper delimitation of the spaces allows for avoiding distortions when transitioning across volumes (e.g., phalanges, these distortions can potentially introduce audible artefacts during the haptic rendering). Then, to build the space partitioning trees (referred to them as tree TemplateTree and tree LeapMTree). Then the boundary of the template and the Leap Motion spaces are defined. Specifically approximated these as cuboids, (15 cuboids to represent phalanges and palm volumes, as in FIG. 3).

This geometry provides a basic structure of the trees.

Let VTi={p0T, p01T, p2T, p3T}⊆TemplateTree and VLMi={p0LM, p1 LM, p2 LM, p3 LM}⊆SLeapMTree, with i∈[1,15]⊆ [symbol] , describe each of the 15 equivalent cuboids in both spaces. The mapping between boundary points is computed as

$${p}_{jLeapM} = {M}_{LeapM}^{Template} * {p}_{jTemplate} , j ∈ \{ 0 , 1 , 2 \}$$

(i.e. vertices to define the axis vectors). 15 cuboids are used to produce the two basic tree structures for TemplateTree and LeapMTree, with each tree containing 15 nodes and each cuboid node VTemplatei in TemplateTree having an analogous cuboid node VLeapMi in LeapMTree.

Any point pTemplate from the current sensation will be inside a unique leaf cuboid node VTemplatei in TemplateTree. Thus, point pTemplate can be mapped to space LeapM using VLeapMi, as in Equation 2.

1.b. Cylinder Approximation for Sensation Relocation in 3D Based on Multi-Space Mapping

This approach makes the sensation move over the 3D hand model surface. It relocates the position of the sensation from the multi-space approach to the 3D hand at any given time in world coordinates. This means this method will work with continuous coordinates for the hand model.

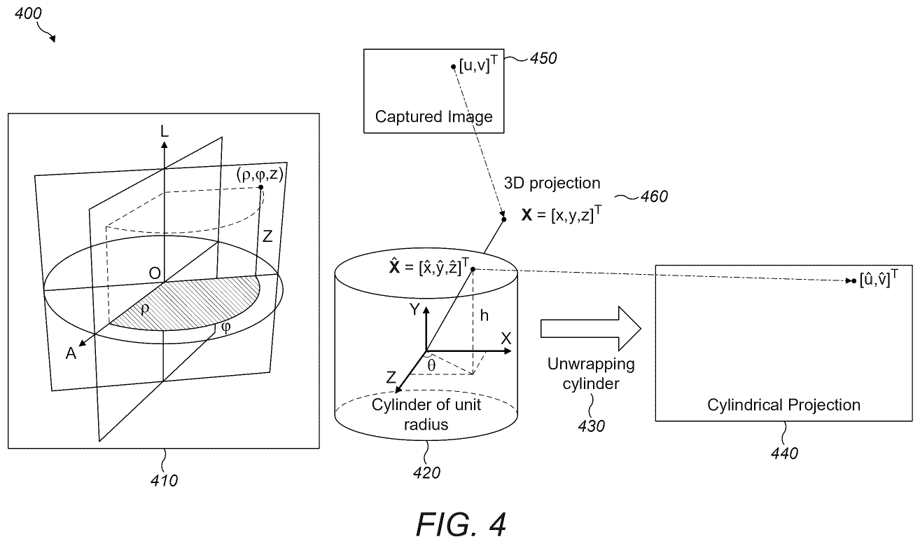

Turning to FIG. 4, shown is a schematic 400 with a cylindrical coordinate system 410 with an origin O, polar axis A and a longitudinal axis L. The dot is the point, for example, with the radial distance ρ=4, angular coordinates φ=130 and high z=4 [2].

This approach takes the local spaces 450 defined for each phalange and palm and computes a cylindrical projection 460 420 430 400 for the sensation plane (located at bone level in the 3D model) over the 3D hand model. For instance, it takes the proximal phalange of the index finger and assumes a cylinder and using a cylindrical projection generates a map between the haptic rendering plane (at bone level) and the cylinder surface (the phalange surface, using the equations 3, 4, 5 and 6).

In this cylindrical projection method, a generic 2D pixel of an acquired image [u, v], can be projected to a 3D point x=[x, y, z] using a camera's intrinsic projection parameters-focal length, f, and optical center [cx, cy] like:

$$[ \begin{matrix}\hat{x} \\ \hat{y} \\ \hat{z}\end{matrix} ] = \frac{1}{\sqrt{{x}^{2} + {z}^{2}}} [ \begin{matrix}x \\ y \\ z\end{matrix} ] = [ \begin{matrix}sin θ \\ h \\ cos θ\end{matrix} ] \qquad ( 3 )$$

$$[ \begin{matrix}{u}^{\text{?}} \\ {v}^{\text{?}}\end{matrix} ] = [ \begin{matrix}f θ + {c}_{x} \\ fh + {c}_{y}\end{matrix} ] \qquad ( 4 )$$

$$x = [ \begin{matrix}x \\ y \\ z\end{matrix} ] = λ {K}^{- 1} [ \begin{matrix}u \\ v \\ 1\end{matrix} ] = λ [ \begin{matrix}\frac{\text{?}}{\text{?}} \\ \frac{\text{?}}{\text{?}} \\ 1\end{matrix} ] \qquad ( 5 )$$

$$\text{?} \text{indicates text missing or illegible when filed}$$

Where K represents the internal calibration matrix of the camera and refers to the pixel's depth. This 3D point is projected onto a unit cylinder as follows:

$$[ \begin{matrix}{x}^{\text{?}} \\ {y}^{\text{?}} \\ {z}^{\text{?}}\end{matrix} ] = \frac{1}{\sqrt{{x}^{2} + {z}^{2}}} [ \begin{matrix}x \\ y \\ z\end{matrix} ] = [ \begin{matrix}sin θ \\ h \\ cos θ\end{matrix} ] \qquad ( 6 )$$

$$\text{?} \text{indicates text missing or illegible when filed}$$

Similar to the 2D to 3D mapping proposed in 1 (multi-space approach), a bijective mapping is generated between the haptic rendering plane and the 3D cylinders composing the finger model surface, allowing for smooth haptic displacement over the 3D hand model, extending the limits of the current sensation rendering by allowing wrap the sensation around the finger reaching the sides of the fingers from the same sensation template in JSON format which is not possible with the current single-plane or multi-space approaches.

### 2. Solution 2: Direct Skinning Approach for Sensation Relocation (Sensation as a Texture)[2]

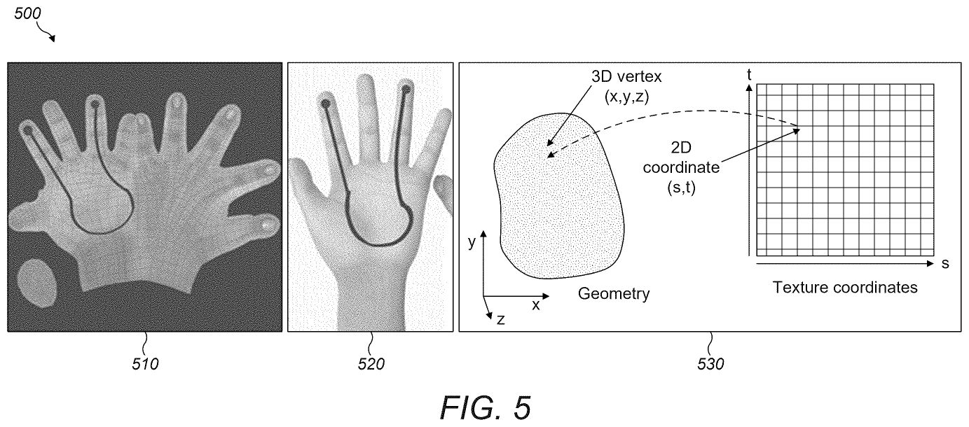

Turning to FIG. 5, shown is a schematic 500 of a 2D Texture of a hand will be generated including the sensation path 510 that will be wrapped on the 3D hand model allowing it to transform from the texture coordinates to the hand model coordinates 520. On the right, the image 530 represents the texture-to-mesh approach.

This approach generates a 2D texture out of the haptic sensation from the sketch-based sensation editor tool (storing the sensation path data in texture coordinates). These texture coordinates can be used to compute the 3D location of the sensation over time by binding the texture to the 3D hand model interpolating the sensation position over time and directly retrieving 3D hand coordinates based on the UV coordinates of the sensation texture at any given time t (see FIG. 5). This means that the solution works with continuous coordinates for the hand model (local-to-hand). This approach does not require the mapping proposed in the solution 1 (multi-space).

This approach uses a single 3D point-to-pixel correspondence, which means that each pixel will be mapped to a single position on a triangle on the hand mesh (avoiding the one-to-many approach commonly used in texture mapping).

The approach takes a given UV coordinates of a texture point and iterates through the triangles in the hand mesh until it finds the one that contains the target point, the triangle vertices are interpolated to find the exact 3D point in the mesh. Since mesh coordinates are in local space, it is important to make sure to convert the points to world space by a local-to-world transformation matrix.

This approach can be embedded into a shader to optimize the per-vertex computation in the 3D position retrieve method.

### Noise Consideration With Skinning-Like Approaches (1.a, 2 & 3)

One concern that may arise from skinning is that the distance between two subsequent points of interest in the model space could be stretched greatly in the real space.

Per prior studies in [3], the maximum hand spread is measured from the outer border of the tip of the little finger to the outer border tip of the thumb. The fingers and thumb are stretched as widely apart as the person finds comfortable.

Table 1 shows hand spread data classified by country and sex.

TABLE 1

| | Mean | | 5% ile | 95% ile | |
|---|---|---|---|---|---|
| Country/Sex | mm | SD | mm | mm | Source |
| UK M | 212.9 | 18.5 | 182.4 | 243.4 | PeopleSize 1998 |
| UK F | 200.2 | 15.6 | 174.6 | 226.9 | PeopleSize 1998 |
| Japan F | 186.5 | 11.1 | 168.2 | 204.7 | PeopleSize 1998 |
| Sri Lanka M | 206 | 15.19 | 185 | 222 | Abeysakara & |
| | | | | | Shahnauvaz |
| | | | | | 1997 |
| Sri Lanka F | 184 | 15.82 | 160 | 210 | Abeysakara & |
| | | | | | Shahnauvaz |
| | | | | | 1997 |
| US M | 213.6 | 18.8 | 182.6 | 244.5 | PeopleSize 1998 |
| US F | 201.1 | 17.0 | 173.1 | 229.0 | PeopleSize 1998 |

Per prior studies in [3], hand breadth (including the thumb) is measured across the palm of the hand at the level of the base of the thumb and including the joint at the base of the thumb.

Table 2 shows hand breadth data classified by country and sex.

TABLE 2

| | Mean | | 5% ile | 95% ile | |
|---|---|---|---|---|---|
| Country/Sex | mm | SD | mm | mm | Source |
| UK M | 106.8 | 5.7 | 97.4 | 116.2 | PeopleSize 1998 |
| UK F | 91.9 | 5.6 | 82.7 | 101.1 | PeopleSize 1998 |
| China M | 102.8 | 6.1 | 92.8 | 112.9 | PeopleSize 1998 |
| China F | 89.5 | 5.6 | 80.2 | 98.7 | PeopleSize 1998 |
| Germany M | 107 | | 98 | 116 | DNN 1986 |
| Germany F | 92 | | 82 | 101 | DNN 1986 |
| Japan M | 105.6 | 4.3 | 98.5 | 112.7 | PeopleSize 1998 |
| Japan F | 89.8 | 4.9 | 81.8 | 97.8 | PeopleSize 1998 |
| Poland M | | | 95 | 114 | PKN 1988 |
| Poland F | | | 82 | 100 | PKN 1988 |
| Sri Lanka M | 99 | 6.53 | 90 | 110 | Abeysakara & |
| | | | | | Shahnauvaz |
| | | | | | 1997 |
| Sri Lanka F | 89 | 5.59 | 80 | 99 | Abeysakara & |
| | | | | | Shahnauvaz |
| | | | | | 1997 |
| US M | 107.1 | 5.8 | 97.6 | 116.7 | PeopleSize 1998 |
| US F | 92.3 | 6.1 | 82.3 | 102.3 | PeopleSize 1998 |

Let's assume the case where a haptic line is drawn from the tip of the thumb to the tip of the pinky finger. The distance of interest thus becomes the breath of the hand in model space, the fingers could be close, and that distance would be equivalent to the hand breath including the thumb and be on average 106.8 mm for a UK male. The worst-case scenario would then be if in the real space, the fingers could be open, and that distance would be equivalent to the hand maximum spread and be on average 212.9 mm for a UK male. The ratio between the two distances in model space and real space is slightly greater than 2. This ratio remains approximately the same across gender and nationality.

Our first proposition is to limit this ratio by using a model space that is midway through the closed fist and open hand/open finger. Thus, the distance could only be shrink/increase by a factor sqrt(2)=0.41. To illustrate this with the previous example, this represents rendering a haptic line at 8 m/s in model space at f=8/0.1598=50Hz (159.8 mm would be the midpoint between 106.8 mm and 212.9 mm) and the two opposite worst case of skinning drawing a line at f=8/0.1068=74.9Hz or f=8/0.2129=37.47Hz.

However, this is assuming skinning at the node level (using hap-e notation). Where the only two coordinates that are modified are the two extremities of the line. Our second proposition is to apply skinning on the sample path of the haptics, where the sampling rate is the one of the array (i.e. 40 kHz or above).

Still, with the example of our line, the space between fingers in the model space is about 53 mm (i.e., (maximum hand spread-hand breath)/2). Thus approximately 13.25 mm between each finger. In comparison, the distance between one finger edge to the other is about 21.36 mm (i.e., hand breadth/5 fingers). Assuming again an 8 m/s line (i.e. 50 Hz), there should be a control point every 0.2 mm. If skinning is then applied, to all these points, only the point between the fingers gets stretched out/reduced.

In the case of a fully open hand, the space between each finger grows from 13.25 mm to 26.5 mm (max spread-hand breadth)/4). This represents a factor 2. Thus, the gap between each control point would also grow by a factor of 2, from 0.2 mm to 0.4 mm. Because the wavelength of ultrasound is about 8 mm (for a 40 khz ultrasound) the jump between consecutive positions is still far less than that of the focal point size itself and poses little impact on audio noise.

In the case of a closed hand, the space between each finger would actually shrink from 13.25 to 0. Similarly, the gap between each control point would also shrink to 0. Thus, no impact on noise as the point would be static.

Thus, using a fully open hand as model space is proposed to remove any audible noise.

### 3. Solution 3: Direct 3D to 3D Mesh Mapping

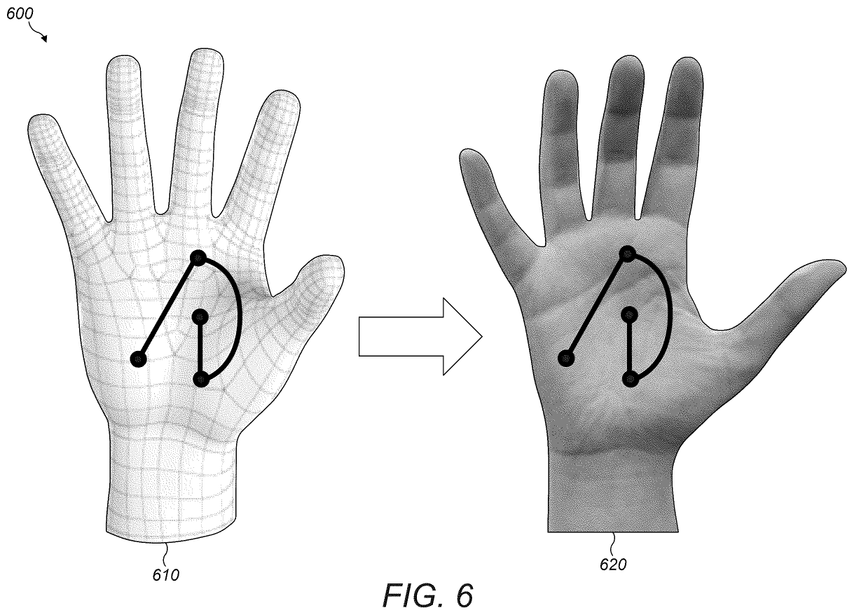

Turning to FIG. 6, shown is a schematic 600 using a 3D model as a template to design haptic sensations. This method can store the sensation pattern in model coordinates (directly in 3D) 610 and automatically use them inside a game engine (e.g. Unity) as local to object coordinates 620.

The skinning-like approaches proposed in this document translate sensation points from 2D to 3D coordinates local to the hand model used by the leap motion tracking system to represent the real hand position. A simple method to directly access the 3D model coordinates of any sensation point is by storing the sensation data in mesh coordinates straight from the design stage inside the sketch-based sensation editor tool. By using a 3D model of the hand as a template in the sensation editing tool instead of the 2D template currently used, will allow not only a direct design of the sensation in a 3-dimensional perspective (a more realistic design pattern) but also it allows to store the sensation's path data in model coordinates which can be directly mapped to a 3D hand model in a virtual reality scenario as a local to model coordinates. See FIG. 6.

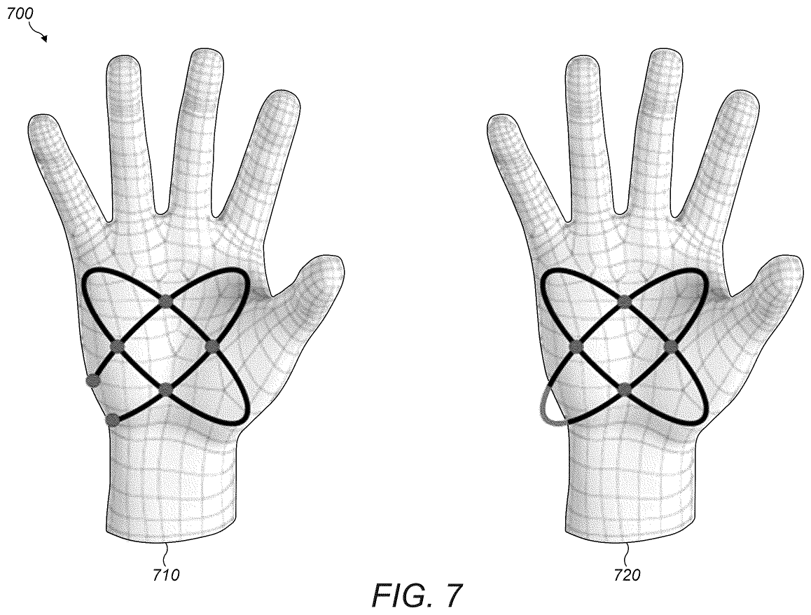

Turning to FIG. 7, shown is a schematic 700 of two methods to handle haptics paths going outside the hand. On the left 710: The lines that go outside the 3D template will be wrapped on the visible side of the 3D model template, where the last intersection point between the haptic line and the 3D template is set as an ending node for the current haptic line. On the right 720: The sections of the haptic lines that go outside the 3D model that were intentionally defined by users to be part of the haptic pattern are dimmed down on the final haptic rendering to avoid unnecessary ultrasound waves being spread out to the environment.

The pattern's sections drawn outside the 3D model are not desired in the final haptic rendering. As shown in FIG. 7, this document considers 2 main mechanisms in this case. 1) Automatic line endings: the haptic lines that go outside the 3 model will be wrapped on the visible side of the 3D model template, the last intersection point between the line and the 3D model template is set as an ending node for the current haptic line. 2) The sections of the haptic lines that go outside the 3D model that were intentionally defined by users to be part of the pattern (e.g. defocusing, hairy skin simulation, passing through the palm/back of the hand) are dimmed down on the final haptic rendering to avoid unnecessary ultrasound waves being spread out to the environment.

This method does not require any additional mapping but only the translation from model coordinates to world coordinates using a standard local-to-world transformation matrix.

### 4. Solution 4: Apply Smooth-Blend Skinning Directly to the Sensation Points

A further approach to the problem is by using the smooth blend skinning algorithm from character animation in computer graphics. This is algorithm is used in computer graphics to bind a mesh-based skin onto a skeleton with discrete bones and joints. The skeleton is represented as a hierarchy of bones, with each discrete joint being considered as the origin of a local transformation at the base of its respective bone. The skin is then created by the artist in the bind pose of the skeleton and in the domain-specific parlance, joint weighting coefficients (or simply weights) are ‘painted’ onto the skin which informs each skin element which joints may modify the skin position. Alternatively, an algorithm may be used to initially create the assignment of weights to the skin, which the artist may then tweak.

Such an algorithm may involve taking the weights to be a normalized distance function either from the joint or bone whose joint represents the base of the hierarchy. Then a function ƒⱼ(V_{bind})=ƒ(r) may be used where r is the shortest distance to the j^{th} base joint (point) or base bone (line segment) in the skeleton to map a general position vector v_{bind} representing a location in the bind pose space to a function of distance from an element in the skeletal hierarchy. This then creates a bijective mapping between arbitrary points from the 3D space of the bind pose of a skeletal model to the 3D space of the skeletal model when the skeleton of the 3D model is posed. For the general point, this may be represented as:

$${w}_{j , q} = {f}_{j} ( {v}_{q , bind} ) ,$$

where a q^{th} point is mapped to a weight associated with the j^{th} hierarchical element. In this disclosure, it is proposed that this algorithm may be repurposed to instead of assigning initial weight values to a mesh skin, instead assign final weighting value to sensation points. The general position vector representing each sensation point created in the bind pose of the skeletal model V_{bind} may then be taken to the final vertex in the 3D space of the posed model vₚₒₛₑ using the equation:

$${v}_{q , pose} = \frac{{\sum }_{j = 1}^{N} {w}_{j , q} ( {M}_{j} * {v}_{q , bind} )}{{\sum }_{j = 1}^{N} {w}_{j , q}}$$

where Mⱼ is the pose transformation associated with the j^{th} hierarchical element. Analogies may be at this stage made between the character design bind space and a sensation design “bind” space, with the posed space of the skeletally modelled character being equivalent to a skeletally tracked body part onto which a sensation may be projected by applying this bijective mapping to move from the space in which the sensation was designed to the space in which the sensation is applied to a posed skeletally tracked body part.

Generation of a bijective mapping between the template (2D) and hand spaces (3D)->Multi-plane approach: This will allow users of the sensation editor tools (sketch-based UI), to generate sensations for the whole hand (volar and dorsal sides of the hand) while improving accuracy on the haptic sensation presentation over the 3D hand, allowing also, more dynamic/complex sensation designs and overcoming the current rendering size/accuracy. The bounding approach will take care of the possible artefacts when transitioning across volumes (e.g., phalanges) by using continuous coordinates in world space.

Generation of a bijective mapping between the template (2D) and hand spaces (3D)->Cylinder approximation: This will allow users of the sketch-based sensation editor tool to generate sensations covering not only the hand palm side when using the standard 2D template from the UI, but also reaching the sides of the fingers and the palm side areas as well (which is not possible with the current single-plane or multi-space approaches). Improving also the presentation accuracy of the mid-air haptic stimulation on the hand. This method also uses continuous coordinates in world and local-to-hand spaces.

Points of Novelty include:
1. Direct skinning approach for sensation relocation (Sensation as a texture): This method provides all the benefits of the other solutions proposed in this document without the need for complex mapping computation inside the game engine (e.g., Unity).
2. Direct 3D to 3D mesh mapping: This approach can gather the benefits from previously proposed methods (multi-space and cylindrical projections) but takes a different path (2D sensation to texture->texture to hand model). It can also potentially reduce the audible artefacts when rendering sensations that move across areas in the hand by using hand model coordinates directly.
3. A smooth-blend skinning directly to the sensation points: This approach combines the benefits of “bijective mapping between the template (2D) and hand spaces (3D)” and “Direct skinning approach for sensation relocation” by directly working the sensation pattern on the template skin coordinates, but local to the bone structure. Improving not only congruency and accuracy between the stimulus designed and the one displayed, but also reducing computation time by precomputing part of the required transformation for it to work in real-time.

### REFERENCES

[1] Hasti Seifi, Sean Chew, Antony James Nascè, William Edward Lowther, William Frier, and Kasper Hornbæk. 2023. Feellustrator: A Design Tool for Ultrasound Mid-Air Haptics. In Proceedings of the 2023 CHI Conference on Human Factors in Computing Systems (CHI '23). Association for Computing Machinery, New York, NY, USA, Article 266, 1-16. https://doi.org/10.1145/3544548.3580728
[2] Pahwa, Ramanpreet Singh, Wei Kiat Leong, Shaohui Foong, Karianto Leman, and Minh N. Do. “Feature-less stitching of cylindrical tunnel.” arXiv preprint arXiv: 1806.10278 (2018).
[3] Laura’ Peebles and Beverley. “Handbook of adult anthropometric and strength measurements” Norris Institute for Occupational Ergonomics, Department of Manufacturing Engineering and Operations Management, University of Nottingham, University Park, Nottingham, NG72RD.

### Conclusion

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. The term “coupled” as used herein is defined as connected, although not necessarily directly and not necessarily mechanically. A device or structure that is “configured” in a certain way is configured in at least that way but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. A method comprising:
scanning a hand having a palm and a plurality of phalanges;
defining a plurality of local spaces for the palm and the plurality of phalanges;
for each of the plurality of local spaces, computing a cylindrical projection for a sensation plane over a 3D hand model.
